(19) United States
(12) Patent Application Publication
(10) Pub. No.: US 2025/0128097 A1
(43) Pub. Date: Apr. 24, 2025

(54) SYSTEM AND METHOD FOR SHARPENING THE FOCAL VOLUME OF THERAPEUTIC AND IMAGING SYSTEMS

(71) Applicant: UNIVERSITY OF UTAH RESEARCH FOUNDATION, Salt Lake City, UT (US)

(72) Inventor: Jan Kubanek, Salt Lake City, UT (US)

(21) Appl. No.: 18/869,772

(22) PCT Filed: Apr. 25, 2023
(86) PCT No.: PCT/US2023/019759

§ 371 Date: Nov. 26, 2024

Related U.S. Application Data
(60) Provisional application No. 63/334,277, filed on Apr. 25, 2022.
(60) Provisional application No. 63/432,344, filed on Dec. 13, 2022.

(51) Int. Cl. A61N 7/02; A61N 7/00

(52) U.S. Cl. CPC A61N 7/02; A61N 2007/0078

(57) ABSTRACT

A method and system that substantially sharpens the depth of focus. The method includes selecting a group of frequencies where the group of frequencies include a plurality of unique frequencies, assigning one of the frequencies in the group of frequencies to two or more of a plurality of transducers, driving the plurality of transducers to generate a plurality of beamlets where each beamlet includes a wave, and emitting the plurality of beamlets toward the target thereby generating an ultrasound field with reduced focal volume. The improvement in the focal volume using this method can comprise a factor of 10 or more, depending on the frequency bandwidth available to the system. The method, which can be applied for diagnostic and therapeutic applications, is entirely non-invasive and does not require labeling or a modification of elements or objects within the target space.

### CROSS-REFERENCE TO RELATED APPLICATIONS

This application is a non-provisional of and claims the benefit of U.S. Provisional Patent Application No. 63/334,277, filed on Apr. 25, 2022 and U.S. Provisional Patent Application No. 63/432,344, filed on Dec. 13, 2022, the entire content of both applications incorporated herein by reference.

### STATEMENT REGARDING FEDERALLY SPONSORED RESEARCH OR DEVELOPMENT

This invention was made with government support under NS100986 awarded by the National Institutes of Health (NIH). The government has certain rights in the invention.

### BACKGROUND

Systems that emit electromagnetic or sonic waves have powered critical diagnostic and interventional applications. A defining feature of many of these systems, including radar and ultrasonic transducers, is that their dimensions are limited by spatial or hardware constraints. In particular, a dimension D of the transmitting aperture is often relatively small with respect to the distance f within which the system operates. The limited aperture size has led to a fundamental problem of an elongated depth of focus. This problem is severe because the depth of focus is proportional to

$${( \frac{f}{D} )}^{2} .$$

Therefore, a reduction in the aperture size leads to a squared increase in the length of the focal region.

Optical imaging systems have overcome this problem by using opposing objective methods with increased aperture size or methods that label or otherwise alter the imaged target or region. However, increasing the aperture size of the system or labeling the targets can be impractical or impossible, especially in domains other than optical imaging. Moreover, interventional or therapeutic applications require a wave-based minimization of the focal volume to specifically manipulate the desired target while sparing surrounding regions.

Accordingly, a method that substantially sharpens the depth of focus without increasing the aperture size of the system would be desirable.

### SUMMARY

To address this fundamental problem, the disclosure provides a method that substantially sharpens the depth of focus for limited apertures. The method is related to opposing objective methods in that the method uses two opposing apertures but does not require an increase in aperture size. Instead, the method described herein tightens the focal region by superimposing a range of frequencies in space and time as shown in FIG. 1. This multifrequency superposition is referred to as MFS. MFS is a practical solution that is also applicable to systems with limited bandwidth. An example below describes an implementation of MFS in hardware and confirms a substantial reduction of the focal volume in an ultrasonic system with limited bandwidth. The method is frequency-independent, which enables applications in ultrasonics, infrasonics, acoustics, radar, and optics and laser.

In one embodiment, the disclosure provides a method for sharpening a focal volume of a therapeutic system or an imaging system. The method comprises applying a plurality of arrays to a target, each array including a plurality of transducers, selecting a group of frequencies, the group of frequencies including a plurality of unique frequencies, assigning one of the frequencies in the group of frequencies to two or more of the plurality of transducers, driving the plurality of transducers to generate a plurality of beamlets, each beamlet including a wave of one of the frequencies in the group of frequencies, and emitting the plurality of beamlets toward the target thereby generating a field of reduced focal volume, wherein the focal volume is improved multifold. In some embodiments, the focal volume is improved by a factor of 10 or more.

In another embodiment, the disclosure provides a therapeutic system comprising a plurality of arrays, each array including a plurality of transducers and a controller electrically coupled to the plurality of transducers. The controller is configured to select a group of frequencies, the group of frequencies including a plurality of unique frequencies, assign one of the frequencies in the group of frequencies to two or more of the plurality of transducers, drive the plurality of transducers to generate a plurality of beamlets, each beamlet including a wave, and emit the plurality of beamlets toward a target thereby generating a focused beam, wherein a focal volume of the beam is improved multifold, and the improvement scales with a frequency bandwidth of the system. In some embodiments, the focal volume of the beam is improved by a factor of 10 or more.

Other aspects of the disclosure will become apparent by consideration of the detailed description and accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates a label-free sharpening of focal field by emitting waves at distinct frequencies and at controlled times. In traditional emission beams (left), a superposition of waves of a single frequency leads to an elongated beam (solid black line). MFS (right) uses multiple frequencies emitted at times such as to amplify destructive interference outside the target, thus sharpening focus. The target is represented by the black dot.

FIG. 2 illustrates the performance of MFS. Simulated (a) and measured (b) fields provided by the traditional single-frequency emission from a single aperture (left column), single-frequency emission from opposing apertures (middle column), and MFS (right column). The corresponding focal volumes were quantified using the bars on the bottom (mean±s.d.).

FIG. 3 illustrates that MFS generates sharper focus than the highest available frequency alone. Left: The field produced by the highest frequency available within the MFS bandwidth. Right: MFS. Error bars represent the s.d. Both fields were obtained using simulations analogous to those of FIG. 2 (at a).

FIG. 4 graphically illustrates that MFS gains as a function of available bandwidth. Mean±s.d. focal volume as a function of fractional bandwidth, relative to the single frequency case (0% bandwidth). The data are provided separately for simulations for each datapoint (black; n=7), and the measurement of FIG. 2 (at b (green)). The black dotted line represents an exponential fit to the data (f(x)=e^{-0.06 x}).

FIG. 5 illustrates fields and waveforms at target for all measured frequency combinations. (a) Same hardware and approach as in FIG. 2 (at b), but now separately (rows) for 1, 3, 5, 10, and 252 frequency components (equally spaced between 500 to 800 KHz) and separately for the X (left column) and Y (middle column) dimensions of the fields. (b) The waveforms at target that result from the superposition of the particular number of frequencies.

FIG. 6 is a block diagram of a system for applying an ultrasonic stimulus according to an embodiment of the present disclosure.

FIG. 7 illustrates a geometry of an array used in the system shown in FIG. 6 according to an embodiment of the present disclosure.

FIG. 8 is a flow chart of a method for sharpening focal volume in the imaging system of FIG. 6 according to an embodiment of the present disclosure.

### DETAILED DESCRIPTION

Before any embodiments are explained in detail, it is to be understood that the embodiments are not limited in application to the details of the configuration and arrangement of components set forth in the following description or illustrated in the accompanying drawings. The embodiments are capable of being practiced or of being carried out in various ways. Also, it is to be understood that the phraseology and terminology used herein are for the purpose of description and should not be regarded as limiting. The use of “including,” “comprising,” or “having” and variations thereof are meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings.

In addition, it should be understood that embodiments may include hardware, software, and electronic components or modules that, for purposes of discussion, may be illustrated and described as if the majority of the components were implemented solely in hardware. However, one of ordinary skill in the art, and based on a reading of this detailed description, would recognize that, in at least one embodiment, the electronic-based aspects may be implemented in software (e.g., stored on non-transitory computer-readable medium) executable by one or more processing units, such as a microprocessor and/or application specific integrated circuits (“ASICs”). As such, it should be noted that a plurality of hardware and software-based devices, as well as a plurality of different structural components, may be utilized to implement the embodiments. For example, “servers” and “computing devices” described in the specification can include one or more processing units, one or more computer-readable medium modules, one or more input/output interfaces, and various connections (e.g., a system bus) connecting the components.

The present disclosure provides a wave-based method to overcome the fundamental issue of elongated beams produced by systems with a limited aperture. As disclosed herein, a method, according to an embodiment, is described that enables operators to use existing ultrasonic hardware to greatly sharpen treatment precision. As demonstrated below, no additional hardware is necessary and the improvement in spatial focus is dramatic. The method can be implemented with many of the emerging ultrasonic therapies of the brain, which all need high spatial precision—neuromodulation, local drug release, and transient opening of the blood brain barrier for delivery of large drugs, genes, or stem cells.

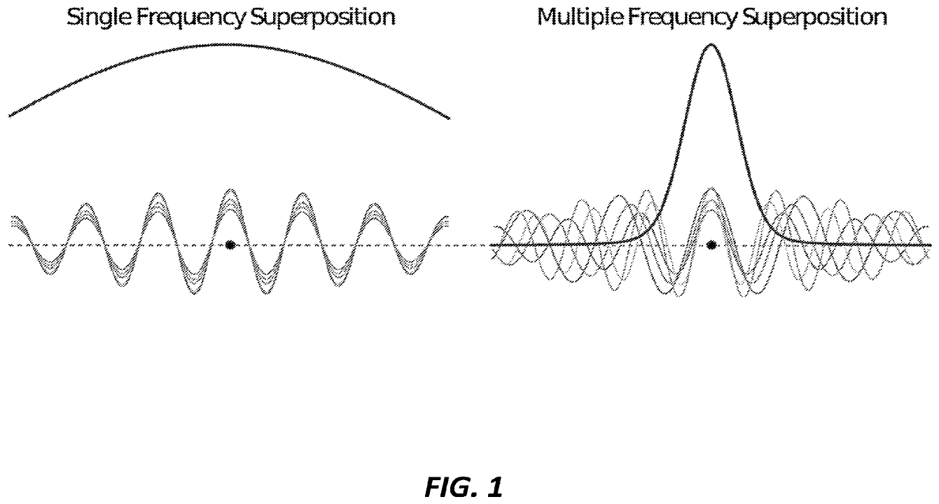

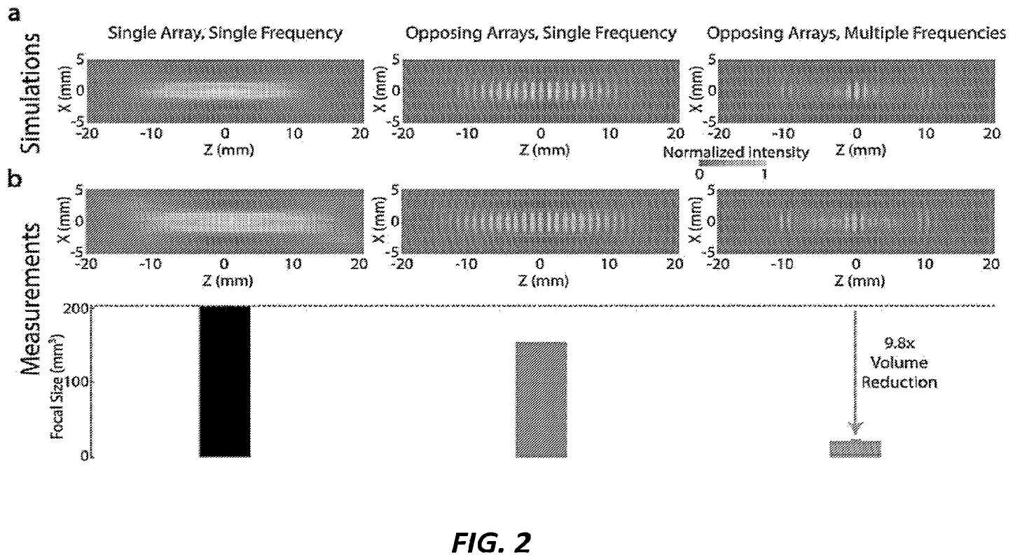

FIGS. 1-2 illustrate the concept of multifrequency superposition (MFS). MFS is a label-free approach and substantially improves the depth of focus of wave-based radiation beams. MFS is based on a controlled superposition of waves and does not require labeling or a modification of elements within the target space. The method was implemented in standard ultrasonic hardware, and it was validated (as discussed below) that the depth of focus can be reduced substantially even for systems with relatively narrow bandwidth.

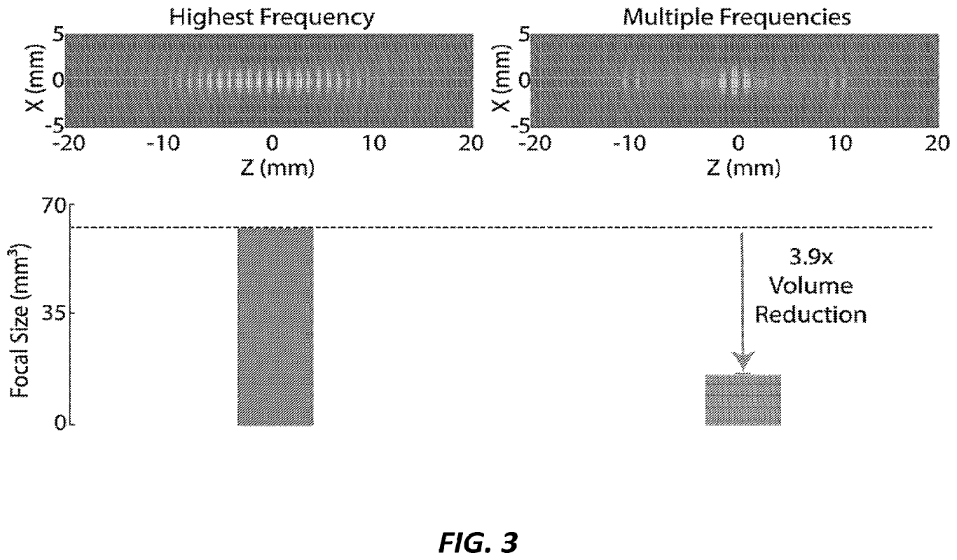

MFS is based on a timed emission of waves to achieve constructive interference at the target of interest. Even a small variation in the frequencies emitted from the individual transducers is sufficient to amplify destructive interference near the target, thus leading to substantial sharpening of the depth of focus. The multifrequency emission is necessary for this effect; using the highest frequency within the bandwidth alone produces a much less focal effect as shown in FIG. 3.

The multifrequency nature of MFS distinguishes it from previous label-free methods. Nonetheless, MFS incorporates an important concept that has been harnessed in optics on several occasions. Specifically, MFS uses two apertures that oppose each other, akin to opposing objective methods in optical imaging. However, unlike in optics, MFS does not require an increase in the aperture or the solid angle to improve the depth of focus. The improvement is achieved for a fixed, limited aperture by emitting waves of multiple frequencies at defined times to achieve constructive interference at the target while amplifying destructive interference elsewhere. For single frequencies, this geometry produces standing waves (FIG. 2). In optics, this effect alone has been harnessed for improving the axial resolution for imaging purposes. MFS goes beyond this step, applying multifrequency superposition to sharpen the focal volume. This way, MFS is also applicable for interventional or therapeutic applications for which the standing-wave pattern itself would not present a notable or desirable property (FIG. 2).

Label-free improvement in spatial focus can also be achieved using superoscillation. Superoscillation applies complex, optimized lenses to focus waves into focal regions whose size evades the Rayleigh criterion. However, the focal benefit comes at the cost of efficiency—the main lobe receives only a few percent of the total energy, while a large portion of energy is dissipated in side lobes. Therefore, although the concept of superoscillation may prove useful for imaging applications, it is unlikely to serve a major role in therapeutic applications. Compared with superoscillation, in MFS, side lobes are smaller than the main lobe (FIG. 2), so the method does not suffer from this issue and is therefore suited also for interventional applications. Furthermore, no lenses are required.

Several previous studies, within ultrasonics, have used multiple frequencies to improve spatial resolution. However, these methods, including frequency compounding in elasticity imaging, apply or receive the individual frequency components in separation. The improvement in spatial resolution follows the standard diffraction-limited resolution, in which sharper focus is obtained using higher frequencies. MFS differs fundamentally from these approaches in that MFS emits the distinct frequency components in a controlled spatiotemporal pattern to achieve a specific superposition pattern at the target.

MFS is particularly useful for interventional and therapeutic applications, which generally require a circumscribed beam. For example, ultrasonic transducers produce a characteristic, cigar-shaped beam. When applied for therapeutic purposes such as thermal or mechanical lesioning, opening of the blood-brain barrier, or neuromodulation, this beam geometry poses a risk of harm to unintended targets. MFS overcomes this limitation (FIGS. 2-3) and can improve the specificity and safety of such treatments. The improvement in the axial resolution may also prove useful in imaging, further increasing axial resolution of existing methods. The increase in axial resolution is expected to be useful for applications that rest on opposing emitters in general. For instance, this method may boost manipulation capabilities of acoustic tweezers or planar linear ion traps.

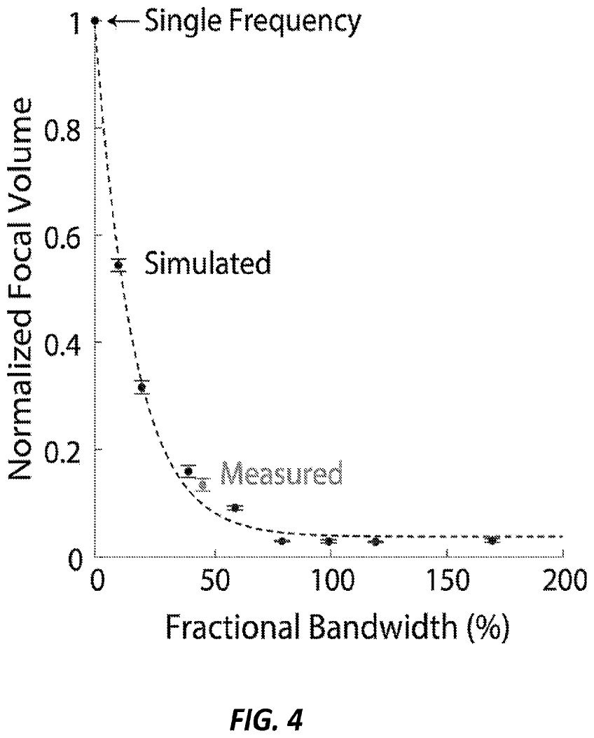

MFS harnesses the available bandwidth of wave-emitting systems. The focal volume improves exponentially with increased bandwidth (FIG. 4). Therefore, even systems with very limited bandwidth may benefit from MFS. In some embodiments, the focal volume is improved multifold. In some embodiments, the focal volume is improved by a factor of 10 or more. Additionally, MFS was implemented using standard ultrasonic hardware, however the method can be also implemented for systems based on electromagnetic waves.

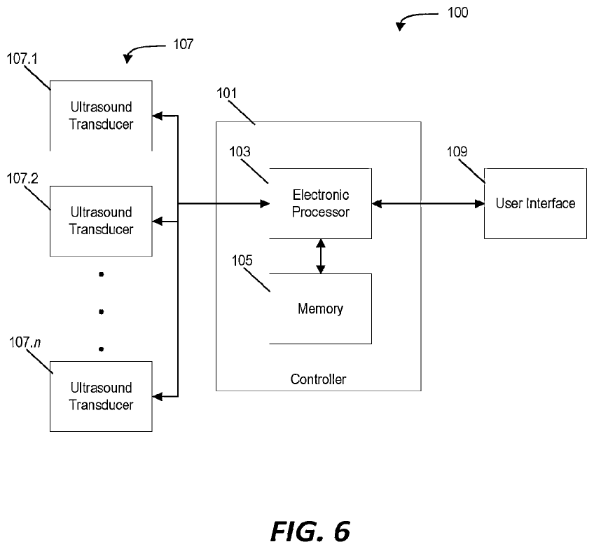

FIG. 6 schematically illustrates an imaging system 100 (e.g., electromagnetic- and sonic-based systems) configured to sharpen a focal volume according to an embodiment of the present disclosure. The imaging system 100 is configured to transmit energy to a target 102 and to acquire (capture) image data (e.g., ultrasound image, MRI image, and the like) from the target 102 (e.g., a patient), in an imaging operation. In one example, the imaging system 100 is embodied as an ultrasound system. The imaging system 100 includes a controller 101, which includes an electronic processor 103 and a non-transitory computer-readable memory 105. The electronic processor 103 is communicatively coupled to the memory 105 and configured to store data to the memory 105 and access stored data from the memory 105. The memory 105 also stores computer-executable instructions that, when executed by the electronic processor 103, provide the functionality of the controller 101 including, for example, the functionality described herein.

Although the example of FIG. 6 illustrates only one memory 105 in other implementations, the system may utilize multiple different memory modules including, for example, a local memory, an external storage device, and/or a remote or cloud-based memory system. Similarly, in different implementations, the system 100 may utilize one or more electronic processors implemented in one or more different computing devices. In some implementations, the controller 101 may be implemented as an application specific controller device while, in other implementations, the controller 101 may be provided as a desktop, laptop, tablet computer, or smartphone. Accordingly, unless otherwise specified, the controller 101 may include one or more computing devices and/or control circuits, one or more electronic processors, and one or more memories.

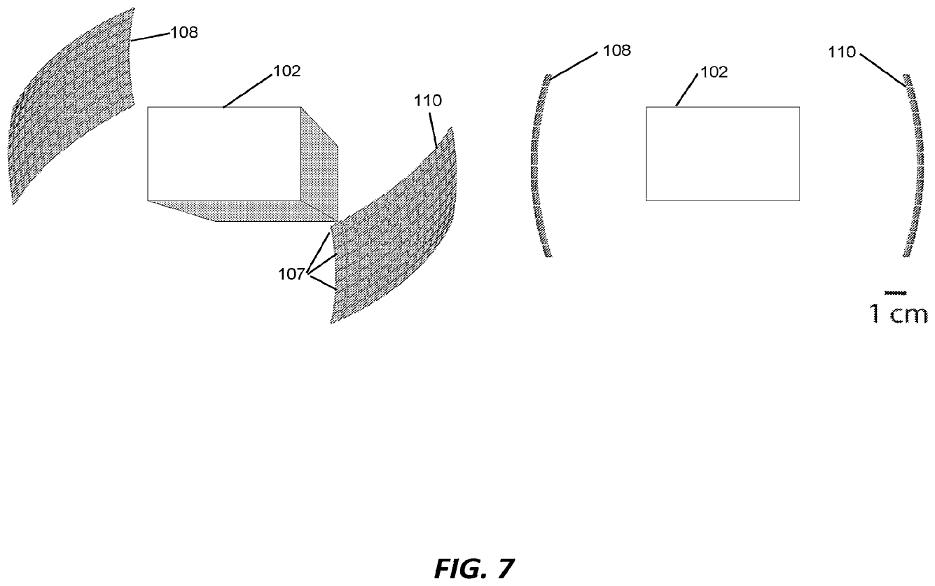

As illustrated in the example of FIG. 6, the controller 101 is communicatively coupled to a plurality of ultrasound transducers 107 including ultrasound transducers 107.1, 107.2, and 107.n. With reference to FIG. 7, the plurality of ultrasound transducers 107 are arranged in one or more arrays, such as a first array 108 and a second array 110. With MFS, a plurality of the transducers 107 emit ultrasound waves so that the individual sound waves arrive at the target 102 in phase, at their peak value. The transducers 107 also emit the sound waves at distinct frequencies (e.g., 500 kHz to 800 kHz). This leads to amplified destructive interference in the vicinity of the target 102 (but not at the target 102).

With continued reference to FIG. 7, each of the arrays 108, 110 are positioned on opposite sides of the target 102. The transducers 107 may be coupled to the target 102 with a hydrogel or standard ultrasound gel. In one example construction, each of the arrays 108, 110 include a spherical curvature with radius of 165 mm with 126 transducers (e.g., 6 mm×6 mm) 107 organized in a 9×14 element grid with inter-element spacing of 0.5 mm. Each array 108, 110 has a height of 55 mm and a width of 86 mm. The ultrasound beam produced by each array 108, 110 has a geometric focus centered at 85 mm away from a face of the array in an axial dimension. The arrays 108, 110 are separated by a distance of 170 mm. In one implementation, the ultrasonic arrays 108, 110 are made of PMN-PT material (e.g., available from Doppler Electronic Technologies, Guangzhou, China), and operated at a center frequency of 650 kHz. The transducers 107 of the arrays 108, 110 are driven by the controller 101 (e.g., available from Vantage256, Verasonics, Kirkland, WA).

As described in further detail below, the controller 101 is configured to selectively and controllably cause the ultrasound transducers 107 in the array(s) to transmit an ultrasound wave and to define/control the parameters of the transmitted ultrasound wave. The controller 101 is also configured to receive output data from other ultrasound transducers 107 in the arrays. In this way, the ultrasound transducers 107 are operated by the controller 101 to transmit and receive ultrasound waves. In some implementations, the controller 101 is configured to electronically communicate with each ultrasound transducer 107 directly while, in other implementations, the controller 101 is indirectly coupled to the plurality of ultrasound transducers 107 through a data acquisition and/or signal routing device (not pictured) that is either incorporated into the controller 101 or provided as a separate additional device.

The controller 101 is also configured to control the operations of other system components of the imaging system 100. The controller 101 includes combinations of hardware and software that are operable to, among other things, control the operation of the system 100, control the output of the transducers 107, etc. The controller 101 includes a plurality of electrical and electronic components that provide power, operational control, and protection to the components and modules within the controller 101 and/or the system 100.

A user interface 109 is included to provide user input to the system 100 and controller 101. The user interface 109 is operably coupled to the controller 101 to control, for example, the output of the arrays 108, 110 (FIG. 7), various ultrasound parameters, etc. The user interface 109 can include any combination of digital and analog input devices required to achieve a desired level of control for the system 100. For example, the user interface 109 can include a computer having a display and input devices, a touch-screen display, a plurality of knobs, dials, switches, buttons, faders, or the like. The controller 101 is configured to analyze or process images or image data collected by the transducer arrays 108, 110 for presentation on a display.

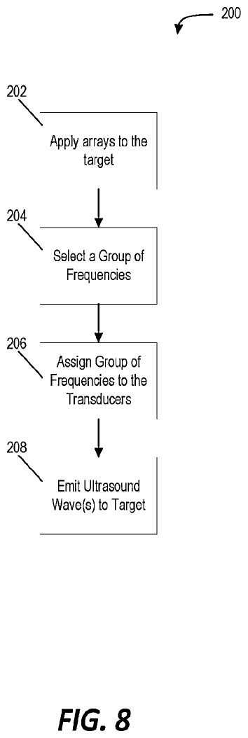

According to an embodiment, the present disclosure provides a method of sharpening focal volume in electromagnetic- and sonic-based systems. FIG. 8 illustrates an example of a method 200 executed by the controller 101 for sharpening focal volume in the imaging system 100. First, at step 202, the arrays 108, 110 are applied to the target 102. Once the arrays 108, 110 are in position, the controller 101 selects (at step 204) a group of frequencies (e.g., 2 frequencies, 3 frequencies, 5 frequencies, n frequencies) for the ultrasound waves that are within the limited bandwidth of the transducers 107. In one implementation, the group of frequencies is different (i.e., each frequency is unique). For example, in a group of two frequencies, the controller selects a first frequency of 500 kHz and a second frequency of 650 kHz. In another example, in a group of five frequencies, the controller selects a first frequency of 500 kHz, a second frequency of 550 kHz, a third frequency of 600 kHz, a fourth frequency of 650 kHz, and a fifth frequency of 700 kHz. In some implementations, the group of frequencies are equally spaced within the bandwidth of the transducers 107. In other implementations, the spacing can be unequal. Next, the controller 101 randomly assigns (at step 206) the group of frequencies to the transducers 107 in the first array 108, and the second array 110. In some implementations, all of the transducers 107 in both arrays 108, 110 are randomly assigned a frequency from the group of frequencies. In other implementations, a subset (i.e., less than all) of the transducers 107 are randomly assigned a frequency from the group of frequencies. Next, the controller 101 drives (at step 208) the plurality of transducers to generate a plurality of ultrasound waves. Lastly, the controller 101 causes all or some of the transducers 107 to emit (at step 210) the ultrasound wave with the assigned frequency to the target 102. This results in a reduced focal volume, which significantly sharpens the focus of the ultrasound beam. Using this method, the focal volume of the ultrasound beam is improved commonly by a factor of 10 for standard ultrasonic hardware (FIG. 2) compared to the focal volume of an ultrasound beam that is delivered to a target not using the method described herein. For systems with broad bandwidth, the improvement can reach a factor of 20 (FIG. 4).

### Example 1—Demonstrating Effective Reduction in Focal Volume with Application of MFS

The MFS concept using simulations that compare the fields produced by MFS with those of traditional, single-frequency approaches was validated. Two spherically focused emitter arrays (as shown in FIG. 6) were used in both simulation and experimental measurements. Each array had a spherical curvature with radius of 165 mm and consisted of 126 individual emitter elements (6 mm×6 mm) organized in a 9×14 element grid with inter-element spacing of 0.5 mm. Each array had a height of 55 mm and a width of 86 mm. The beam produced by each array had a geometric focus centered at 85 mm away from the face of the array in the axial dimension. In the analyses that used opposite configurations, the arrays were facing each other, separated by a distance of 170 mm.

The ultrasonic arrays 108, 110 were made of the PMN-PT material (e.g., available from Doppler Electronic Technologies, Guangzhou, China), and operated at a fundamental frequency of 650 kHz. The individual elements of the arrays were driven by a programmable system (e.g., Vantage256, available from Verasonics, Kirkland, WA).

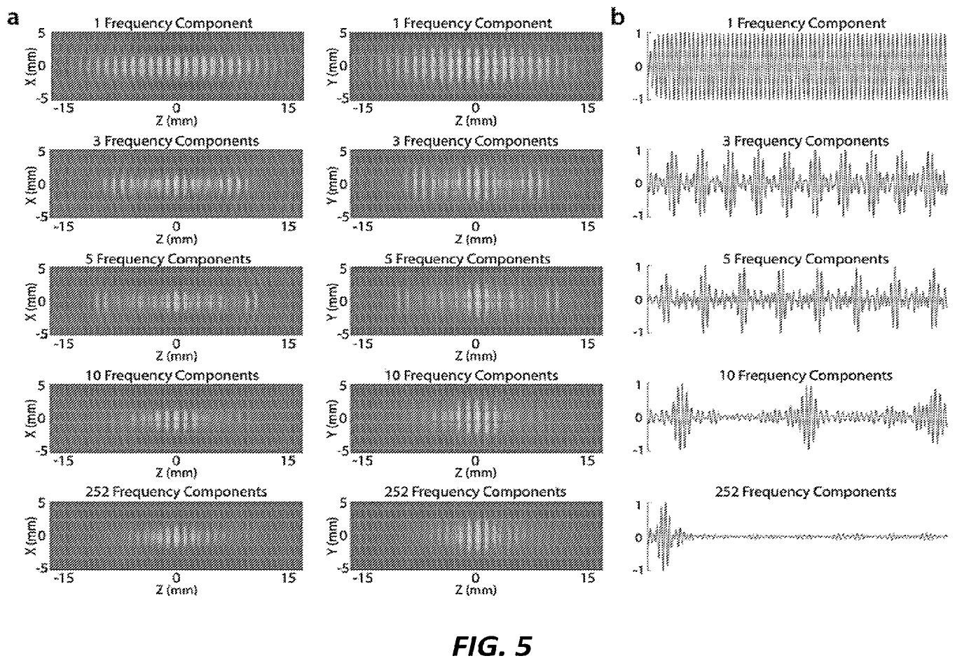

The available bandwidth was discretized into an arbitrarily high number of frequencies. Five sets of frequencies were tested. In all cases, the frequencies were equally spaced across the transducers' bandwidth, which ranged from 500 kHz to 800 kHz. The effects of single frequency (650 kHz), three frequencies (500, 650, 800 kHz), five frequencies, ten frequencies, and 252 frequencies (FIG. 5) were measured. It was found that five frequencies provided a favorable trade-off between sharp focus and the number of necessary frequencies (FIG. 5). Therefore, the simulations and measurements used five frequencies, with the exception of FIG. 4, which used 252 frequencies to fully harness the available bandwidth.

Each element of the array was randomly assigned one frequency from the set. It was found that randomizing the frequency assignment across the array geometry minimizes the focal volume. Moreover, assigning the frequencies to the elements randomly produced multiple realizations and multiple measurements, which were key for statistical valuations (i.e., producing the confidence error bars in all figures).

Each element was driven for 153 s, i.e., the duration of 100 cycles at 650 kHz. For the elements of the actual hardware, the amplitude output was normalized by the frequency characteristic of each element. This way, all frequencies across the 500-800 kHz bandwidth had comparable amplitude.

Ultrasonic transducers require a certain number of cycles to reach maximum amplitude. To take this hardware constraint into account, the transmission of the waveforms was delayed such that their 10th peak arrived at the target at the same time.

The simulations were performed using Field II. The output was recorded over a 10 mm×40 mm grid in the XY and XZ planes with 0.15 mm spacing. The waveform at each point in the grid was recorded and saved. Since field amplitudes are additive, the total pressure was computed as the sum of the contributions of the individual elements.

The ultrasonic pressure fields were measured using hydrophone field scans. Specifically, the fields were measured using a capsule hydrophone (e.g, HGL-0200, available from Onda) secured to 3-degree-of-freedom programmable translation system (e.g., Aims III, available from Onda). In accord with the simulations, the hydrophone scanned both the XZ and YZ planes, each within 10 mm×40 mm in 0.15 mm steps. Compared to the simulations, which computed the resulting field element-wise, during the actual measurements, all transducers were excited at once to produce the total field.

The maximal pressure P over the time of the simulation was registered at each location, and this value was converted into intensity I using

$$I = \frac{{p}^{2}}{2 Z} ,$$

where Z=1.5 MPa is the acoustic impedance of water. The intensity values were peak-normalized in all plots.

The focal volume was quantified by measuring the total size of the intensity field above half the maximum value. Specifically, the convex hull of the voxels just exceeding the half-maximum intensity was used in both the XY and XZ planes. For each position on the x-axis, the full width half max—the width of the focal volume at half-maximum intensity—in the Y and Z dimension were calculated. Then, these products were integrated over the x axis to get the total volume. In particular, let the functions FWHM_{y}(x) and FWHM_{z}(x) denote the full width half max at position x in the Y and Z dimension, respectively. The focal volume then equals ∫FWHM_{y}(x)FWHM_{z}(x)dx.

### Example 2—Validation of MFS Concept in a Therapeutic System

All cases used spherically focused phased arrays of 126 elements as shown in FIG. 7. In the first case, a single frequency (650 kHz) was emitted from a single array. As illustrated in FIG. 2 (at a, left) shows that this traditional approach produces a characteristic elongated beam. The beam had a focal volume of 112.92 mm³.

Next, the effect of opposing apertures was tested. The opposing apertures produced the expected standing wave pattern, and reduced the focal volume by a modest 5.3%, to 106.89 mm³ as shown in FIG. 2 (at a, middle). Applying MFS to the same opposing array geometry reduced the focal volume by 86.0% as illustrated in FIG. 2 (at a, right), to an average 15.82±0.51 mm³ (mean s.d.). Thus, MFS provided a 6.76±0.21 (mean±s.d.) reduction of the focal volume compared with the same geometry not using MFS, and this difference was significant (t₁₉=800.5, p=1.74×10⁻⁴⁴, two-tailed t-test).

These simulations were implemented in ultrasonic hardware and the produced fields were measured using a hydrophone. The resulting fields are shown in FIG. 2 (at b), in the same format as in FIG. 2 (at a). The single array and opposing arrays driven at the single frequency had focal volumes of 203.37 mm³ and 154.47 mm³, respectively. In line with the simulations, MFS reduced the focal volume substantially, to a mere 20.75±1.79 mm³ (mean±s.d.). Thus, compared to the opposing arrays at center frequency, MFS provided a 7.44±0.59 (mean±s.d.) reduction of the focal volume, and this difference was significant (t₁₉=298.09, p=1.03×10⁻²⁹, two-tailed t-test).

MFS used a ±23% bandwidth (500 kHz to 800 kHz) with respect to the central frequency (650 kHz) used by the single-frequency approaches. The improvements in the focal volume were tested to ensure they were not simply due to the presence of higher frequencies (i.e., frequencies over 650 kHz) in the bandwidth. FIG. 3 shows that this was not the case. MFS (shown on the right) reduced the focal volume substantially also with respect to the single-frequency approach operating at the highest available frequency (800 kHz; shown on the left). Specifically, the volume reduced from 106.89 mm³ to 15.82±0.50 mm³ (mean±s.d.), i.e., by a factor of 3.9±1.3, and the difference was significant (t₁₉=408.77, p=6.12×10⁻³⁹, two-tailed t-test).

Next, the MFS improvements of focus scale with the available bandwidth. FIG. 4 shows the focal volumes for fractional bandwidths in the range from 0% to 170%, using simulations (black) and the measurement of the output of the hardware implementation (green). It was found that the focal volume decreases exponentially with the available bandwidth (98% of variance explained in the data points). This exponential effect was favorable in regard to systems with limited bandwidth. For instance, an 80% bandwidth led to a volume that was only 2.9±0.1% (mean±s.d.) of the single-frequency case volume (0% bandwidth). Moreover, a fractional bandwidth of just 10% yielded a 45.6±0.5% relative volume.

Finally, how the MFS effect depends on specific selections of the frequency distribution within the available bandwidth (FIG. 5) was tested. There was a trend toward more frequency values providing a sharper focus (FIG. 5 (at a)), but there was local deviation from this observation e.g., five equally distributed frequencies (FIG. 5 (at a)). As expected, the superposition of frequencies could produce a complex waveform at the target (FIG. 5 (at b)). The more frequency components available, the more impulse-like the waveform at the target, as expected from the Fourier theory. These observations were based on continuous waveforms.

What is claimed is:

1. A method for sharpening a focal volume of a therapeutic system or an imaging system, the method comprising:
applying a plurality of arrays to a target, each array including a plurality of transducers;
selecting a group of frequencies, the group of frequencies including a plurality of unique frequencies;
assigning one of the frequencies in the group of frequencies to two or more of the plurality of transducers;
driving the plurality of transducers to generate a plurality of beamlets, each beamlet including a wave of one of the frequencies in the group of frequencies; and
emitting the plurality of beamlets toward the target thereby generating a field of reduced focal volume, wherein the focal volume is improved multifold.

2. The method of claim 1, wherein the group of frequencies are based on a frequency bandwidth of the system.

3. The method of claim 1, wherein the transducers are of arbitrary dimensions, based on dimension constraints of the system.

4. The method of claim 1, wherein the system is an ultrasound system, and wherein the sinusoidal wave is an ultrasound wave.

5. The method of claim 1, wherein all of the plurality of transducers are assigned one of the frequencies in the group of frequencies.

6. The method of claim 1, wherein the plurality of arrays includes two arrays that are positioned on opposite sides of the target.

7. The method of claim 1, wherein each array in the plurality of arrays includes at least 3 transducers.

8. The method of claim 1, wherein the group of frequencies are equally spaced between a bandwidth of the transducers.

9. The method of claim 1, wherein the group of frequencies are in a range of 500 kHz to 800 kHz.

10. A therapeutic system comprising:
a plurality of arrays, each array including a plurality of transducers; and
a controller electrically coupled to the plurality of transducers, the controller configured to:
select a group of frequencies, the group of frequencies including a plurality of unique frequencies;
assign one of the frequencies in the group of frequencies to two or more of the plurality of transducers;
drive the plurality of transducers to generate a plurality of beamlets, each beamlet including a wave; and
emit the plurality of beamlets toward a target thereby generating a focused beam, wherein a focal volume of the beam is improved multifold, and the improvement scales with a frequency bandwidth of the system.

11. The system of claim 10, wherein the group of frequencies are based on the frequency bandwidth of the system.

12. The system of claim 10, wherein the transducers are of arbitrary dimensions, based on dimension constraints of the system.

13. The system of claim 10, wherein the system is an ultrasound system, and wherein the wave is an ultrasound wave.

14. The system of claim 10, wherein all of the plurality of transducers are assigned one of the frequencies in the group of frequencies.

15. The system of claim 10, wherein the plurality of arrays includes two arrays that are positioned on opposite sides of a target.

16. The system of claim 10, wherein each array in the plurality of arrays includes at least 3 transducers.

17. The system of claim 10, wherein the group of frequencies are equally spaced between a bandwidth of the transducers.

18. The system of claim 10, wherein the group of frequencies are in a range of 500 kHz to 800 kHz.
